(19) United States
(12) Patent Application Publication
(10) Pub. No.: US 2002/0038853 A1
(43) Pub. Date: Apr. 4, 2002

(54) Electron beam exposure apparatus

(75) Inventors: Shinichi Hamaguchi, Tokyo (JP); Hiroshi Yasuda, Tokyo (JP)

Correspondence Address: David L. Fehrman, Morrison & Foerster LLP, 35th Floor, 555 W. 5th Street, Los Angles, CA, 90013, US

(73) Assignee: ADVANTEST CORPORATION

(21) Appl. No.: 09/965,399

(22) Filed: Sep. 26, 2001

(30) Foreign Application Priority Data
Oct. 3, 2000 (JP) 2000-342659

(51) Int. Cl. G21G 5/00; A61N 5/00

(52) U.S. Cl. 250/492.3

(57) ABSTRACT

An electron beam exposure apparatus for exposing a wafer using an electron beam, including: a shaping unit for shaping a cross sectional shape of the electron beam so that the cross sectional shape has a rectangular cross-section that includes a first edge and a second edge, which is substantially perpendicular to the first edge; and a control unit connected to the shaping unit for determining at least one of a length of the second edge and an irradiation time of the electron beam based on a length of the first edge.

### BACKGROUND OF THE INVENTION

1. Field of the Invention

The present invention relates to an electron beam exposure apparatus. More particularly, the present invention relates to an electron beam exposure apparatus that can effectively expose a pattern on a wafer.

2. Description of the Related Art

An electron beam exposure apparatus exposes a pattern on a wafer using an electron beam to manufacture a semiconductor device. As one example of the electron beam to be exposed on a wafer, there is a variable shaped beam, the cross sectional shape of which is rectangular and the size of which can be varied as desired. The electron beam exposure apparatus generates an electron beam and shapes the electron beam to the necessary size and shape and then irradiates the shaped electron beam on a wafer. The conventional electron beam exposure apparatus has a problem such that the cross sectional shape of the electron beam to be exposed on a wafer cannot be effectively determined for the pattern to be exposed on the wafer. Therefore, the conventional electron beam exposure apparatus cannot effectively expose a pattern on a wafer.

### SUMMARY OF THE INVENTION

Therefore, it is an object of the present invention to provide an electron beam exposure apparatus which overcomes the above issues in the related art. This object is achieved by combinations described in the independent claims. The dependent claims define further advantageous and exemplary combinations of the present invention.

According to the first aspect of the present invention, an electron beam exposure apparatus for exposing a wafer using an electron beam comprises: a shaping unit for shaping a cross sectional shape of the electron beam so that the cross sectional shape has a rectangular cross-section that includes a first edge and a second edge, which is substantially perpendicular to the first edge; and a control unit connected to the shaping unit for determining at least one of a length of the second edge and an irradiation time of the electron beam based on a length of the first edge.

The control unit may determine at least one of the length of the second edge and the irradiation time of the electron beam further based on a pattern to be exposed on the wafer. The shaping unit may have a shaping member that includes an opening part which shapes the cross-sectional shape of the electron beam to the rectangular cross-section; and the maximum length of the second edge is limited by the size of the opening part.

The control unit may determine at least one of said length of said second edge and said irradiation time so that a product of a current density of said electron beam, which is shaped in the rectangular cross-section, an area of said rectangular cross-section, and said irradiation time becomes substantially constant. The control unit may determine the length of the second edge so that a product of a current density of the electron beam, which is shaped in the rectangular cross-section, and an area of the rectangular cross-section becomes substantially constant.

The apparatus may further comprise a deflection unit that deflects the electron beam shaped in the rectangular cross-section; and the pattern to be exposed on the wafer in a range where the deflection unit deflects the electron beam, which is shaped in the rectangular cross-section, includes a third edge and a fourth edge, which is substantially perpendicular to the third edge; and the length of first edge is determined based on a length of the third edge and a length of the fourth edge.

A length of an edge along a longitudinal direction of the rectangular cross-section may be limited by the size of the opening part. The control unit may have a means for determining the length of a first edge so that the shape of the rectangular cross-section becomes similar to the shape of the pattern to be exposed on the wafer. The control unit may have a means for determining the length of a first edge to divide the pattern to be exposed on the wafer in a range where the deflection unit deflects the electron beam so that the number of times of irradiating the shaped electron beam becomes minimum.

The apparatus may further comprise a means for generating a plurality of the electron beams; and the shaping unit shapes a cross-sectional shape of each of the electron beams into the rectangular cross-section that includes a first edge and a second edge, which is substantially perpendicular to the first edge; and the control unit determines at least one of each of the length of the second edges and each of the irradiation time of the electron beams.

According to the second aspect of the present invention, a method for exposing a wafer using an electron beam comprises: shaping a cross sectional shape of the electron beam so that the cross sectional shape has a rectangular cross-section that includes a first edge and a second edge, which is substantially perpendicular to the first edge; and determining at least one of a length of the second edge and an irradiation time of the electron beam based on a length of the first edge.

The determining may determine at least one of the length of the second edge and the irradiation time of the electron beam further based on a pattern to be exposed on the wafer. The shaping may shape the cross-sectional shape of the electron beam to the rectangular cross-section by an opening part having an opening, through which the electron beam passed through; and the maximum length of the second edge is limited by the size of the opening part.

The determining may determine at least one of the length of the second edge and the irradiation time so that a product of a current density of the electron beam, which is shaped in the rectangular cross-section, an area of the rectangular cross-section, and the irradiation time becomes substantially constant. The determining may determine the length of the second edge so that a product of a current density of the electron beam, which is shaped in the rectangular cross-section, and an area of the rectangular cross-section becomes substantially constant.

The method may further comprise: deflecting the electron beam shaped in the rectangular cross-section; and the pattern to be exposed on the wafer in a range, where the electron beam shaped in the rectangular cross-section is deflected, includes a third edge and a fourth edge, which is substantially perpendicular to the third edge; and the length of first edge is determined based on a length of the third edge and a length of the fourth edge. A length of an edge along a longitudinal direction of the rectangular cross-section may be limited by the size of the opening part.

The determining may determine the length of a first edge so that the shape of the rectangular cross-section becomes similar to the shape of the pattern. The determining may determine the length of a first edge to divide the pattern to be exposed on the wafer in a range where the electron beam is deflected so that the number of times of irradiating the shaped electron beam becomes minimum.

The method may further comprise: generating a plurality of the electron beams; and the shaping shapes a cross-sectional shape of each of the electron beams into the rectangular cross-section that includes a first edge and a second edge, which is substantially perpendicular to the first edge; and the determining determines at least one of each of the length of the second edges and each of the irradiation time of the electron beams.

This summary of the invention does not necessarily describe all necessary features of the present invention. The present invention may also be a sub-combination of the above described features. The above and other features and advantages of the present invention will become more apparent from the following description of embodiments taken in conjunction with the accompanying drawings.

### BRIEF DESCRIPTION OF THE DRAWINGS

FIG. 1 shows a configuration of an electron beam exposure apparatus according to an embodiment of the present invention.

FIGS. 2A-2C show an example of the cross sectional shape of the electron beam.

FIGS. 3A-3G show an example of an exposure pattern 200 and a cross sectional shape of the electron beam to be exposed on the wafer 44.

### DETAILED DESCRIPTION OF THE INVENTION

The invention will now be described based on the preferred embodiments, which do not intend to limit the scope of the present invention, but exemplify the invention. All of the features and the combinations thereof described in the embodiments are not necessarily essential to the invention.

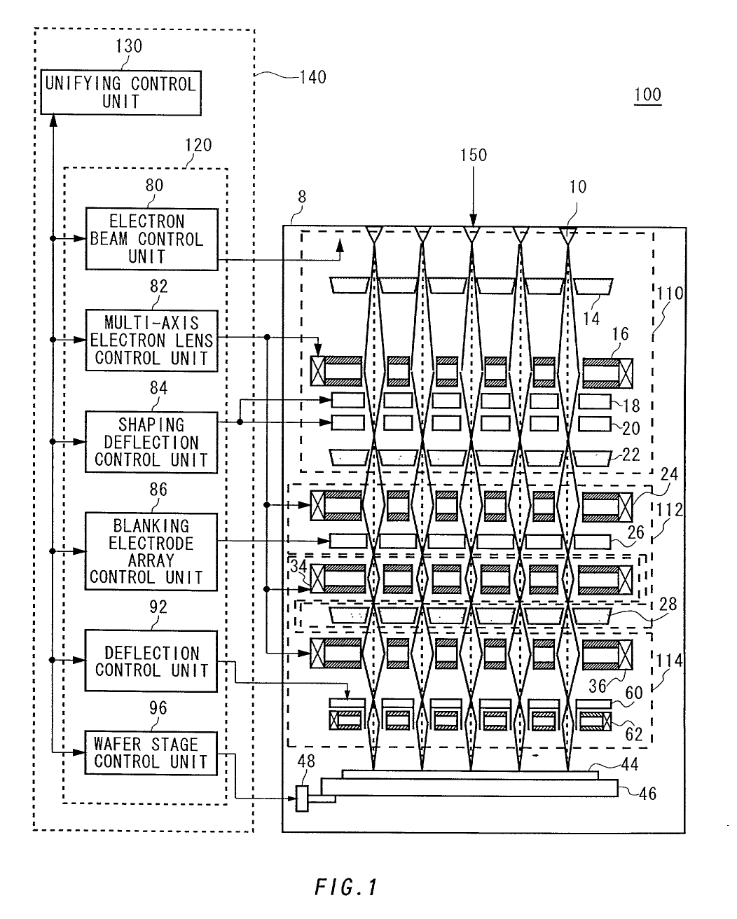

FIG. 1 shows a configuration of an electron beam exposure apparatus according to an embodiment of the present invention. The electron beam exposure apparatus 100 comprises an exposing unit 150 and a control system 140. The exposing unit 150 performs a predetermined exposure process on a wafer 44 by an electron beam. The control system 140 controls the operation of each component included in the exposing unit 150.

The exposing unit 150 comprises an electron beam shaping unit 110, an irradiation-switching unit 112, and electron optics inside a casing 8. The electron beam shaping unit 110 generates a plurality of electron beams and shapes the cross sectional shape of an electron beam into the desired shape. The irradiation-switching unit 112 switches whether or not to irradiate an electron beam on the wafer 44 for each of a plurality of electron beams independently. The electron optics includes a wafer projection system 114 that adjusts a direction and a size of an image of the pattern to be transferred to the wafer 44. Furthermore, the exposing unit 150 comprises a wafer stage 46 and stage system that includes a wafer-stage driving unit 48. The wafer 44, on which the pattern is to be exposed, is mounted on the wafer stage 46. The wafer-stage driving unit 48 moves the wafer stage 46.

The electron beam shaping unit 110 has electron guns 10, a first shaping member 14, a second shaping member 22, a first multi-axis electron lens 16, a first shaping deflection unit 18, and a second shaping deflection unit 20. The electron guns 10 generate a plurality of electron beams. The first shaping member 14 and the second shaping member 22 each have a plurality of opening parts that shape the cross sectional shape of an electron beam by passing the electron beam through an opening part. The first multi-axis electron lens 16 converges each of the plurality of electron beams independently to adjust the focus of the electron beams. The first shaping deflection unit 18 and the second shaping deflection unit 20 deflect each of the plurality of electron beams that pass through the first shaping member 14 independently.

The irradiation-switching unit 112 has a second multi-axis electron lens 24, a blanking electrode array 26, and an electron beam shielding member 28. The second multi-axis electron lens 24 converges a plurality of electron beams independently and adjusts the foci of the electron beams. The blanking electrode array 26 switches whether or not to irradiate the electron beams on the wafer 44 for each beam of the electron beams independently by deflecting each beam of the plurality of electron beams independently. The electron beam shielding member 28 includes a plurality of opening parts, through which the electron beams pass. The electron beam shielding member 28 shields the electron beams deflected by the blanking electrode array 26.

The wafer projection system 114 has a third multi-axis electron lens 34, a fourth multi-axis electron lens 36, a deflection unit 60, and a fifth multi-axis electron lens 62. The third multi-axis electron lens 34 converges a plurality of electron beams independently to reduce the irradiation diameter of each of the electron beams. The fourth multi-axis electron lens 36 converges a plurality of electron beams independently to adjust the focus of each of the electron beams. The deflection unit 60 deflects each of a plurality of electron beams independently to the desired position on the wafer 44. The fifth multi-axis electron lens 62 functions as an object lens for the wafer 44 to converge each of a plurality of electron beams independently.

The control system 140 comprises a unifying control unit 130 and an individual control system 120. The individual control system 120 has an electron beam control unit 80, a multi-axis electron lens control unit 82, a shaping deflection control unit 84, a blanking electrode array control unit 86, a deflection control unit 92, and a wafer stage control unit 96. The unifying control unit 130 unifies and controls each control unit included in the individual control system 120. A work station is one example of the unifying control unit 130. The electron beam control unit 80 controls the electron guns 10. The multi-axis electron lens control unit 82 controls the electric current provided to the first multi-axis electron lens 16, the second multi-axis electron lens 24, the third multi-axis electron lens 34, the fourth multi-axis electron lens 36, and the fifth multi-axis electron lens 62.

The shaping deflection control unit 84 controls the first shaping deflection unit 18 and the second shaping deflection unit 20. The blanking electrode array control unit 86 controls the voltage that is applied to the deflection electrode included in the blanking electrode array 26. The deflection control unit 92 controls the voltage applied to the deflection electrode included in a plurality of deflectors that are included in the deflection unit 60. The wafer stage control unit 96 controls the wafer-stage driving unit 48 and moves the wafer stage 46 to the predetermined position.

The control system 140 has a control means that determines at least one of a length of a second edge of a rectangular cross-section of an electron beam, which is substantially perpendicular to a first edge of the beam, and an irradiation time for irradiating the electron beam on the wafer 44 based on a length of the first edge included in the rectangular cross-section of the electron beam. The control means may be a unifying control unit 130. The control system 140 preferably determines at least one of the length of the second edge and the irradiation time so that a product of a current density of the electron beam, which is shaped in rectangular cross-section, an area of the rectangular cross-section, and the irradiation time becomes substantially constant.

In the present embodiment, the control system 140 determines the length of the second edge so that a product of a current density of the electron beam that is shaped in rectangular cross-section and an area of the rectangular cross-section becomes substantially constant. At this time, the length of the second edge, which is a length in a longitudinal direction of the rectangular cross-section, is limited by the size of the opening part included in the second shaping member 22. Furthermore, the control means preferably determines the length of the first edge for each of the electron beams based on a pattern to be exposed on the wafer by each of the electron beams. The control means then determines at least one of the length of the second edge and the irradiation time based on the length of the first edge for each of the electron beam.

The control system 140 instructs the shaping deflection control unit 84 about the necessary amount of deflection for deflecting the electron beam by the first shaping deflection unit 18 and the second shaping deflection unit 20 in order to shape the cross sectional shape of the electron beam according to the determined length of the first edge and the determined length of the second edge. Furthermore, the control system 140 instructs the blanking electrode array control unit 86 about the irradiation time for irradiating the electron beam on the wafer, the irradiation time of which is determined based on the length of the first edge. In the present embodiment, the irradiation time is a constant time. In the following, the operation of determining the cross sectional shape of the electron beam will be explained.

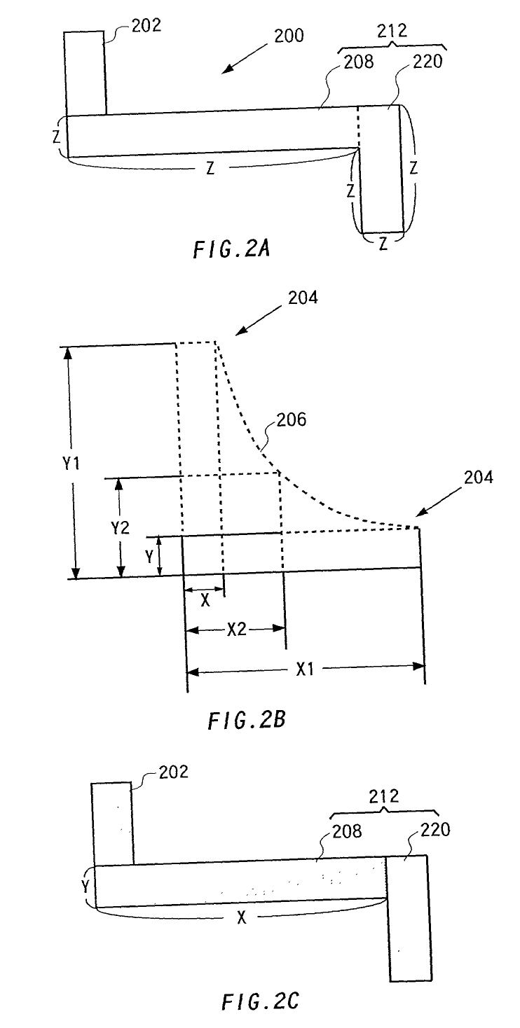

FIGS. 2A-2C show an example of the cross sectional shape of the electron beam, which is to be shaped by the first shaping member 14 and the second shaping member 22. FIG. 2A shows an exposure pattern 200 to be exposed on the wafer 44 by the electron beam. In the exposure pattern 200, the region 202 is a region where the exposure process has already been performed.

FIG. 2B shows a shape of the electron beam, which is to be shaped by the electron beam shaping unit 110 based on the instruction of the control system 140. The control system 140 specifies the region 212 (note FIG. 2A) to be exposed by the electron beam based on the exposure pattern 200. The control system 140 determines the length of the first edge Y, which is to be a standard of the cross sectional shape of the electron beam to be shaped, based on the one of the lengths of the edges Z included in the specified region 212. In the present embodiment, the length of edge Z and the length of the first edge Y are substantially the same. The specified region 212 includes two regions 208 and 220 in FIG. 2A.

Next, the control system 140 determines the length of the second edge X based on the determined length of the first edge Y. The cross sectional shape of the electron beam should have the length of the second edge X, which is determined based on the determined length of the first edge Y. For example, the length of the second edge X can be determined such that the area of the cross sectional shape of the electron beam to be exposed to the region 212 in the exposure pattern 200, and the area of the cross sectional shape of the electron beam determined based on the first edge Y and the second edge X become substantially equal. In other words, the length of the second edge X may be obtained by dividing the area of the cross sectional shape of the electron beam to be exposed to the region 208 or 220 by the length of the first edge Y.

Furthermore, the length of the second edge is preferably determined so that the value of the electric current of the electron beam becomes less than the reference value of the permissible level of the spread of the electron beam along the direction substantially perpendicular to the direction of the irradiation of the electric beam caused by the coulomb force of an electron included in the electron beam.

The range that the vertex 204, which is included in the rectangular cross-section of the electron beam, can take is shown by the curve 206 when the lengths of the first edge and the second edge are determined so that the value of the electric current of the electron beam becomes substantially equal to the reference value. The lengths of the first edge and the second edge are preferably determined such that the vertex 204 is located on the curve 206.

Furthermore, in case the length of the first edge is determined as the length Y2, and the length of the second edge is determined as the length X2 when the length of the first edge and the length of the second edge become substantially equal within some range where the vertex 204 exists on the curve 206, the lengths of the first edge may be determined within the range where the length of the first edge becomes the length X2 or less.

Moreover, in another exposure pattern, the length of the first edge may be determined based on the length of the second edge. In this case, the length of the second edge can be the same as the length X2 or less. In the present embodiment, the length of the second edge is determined by the length of the first edge Y. Furthermore, the length of the second edge is limited to the length X by the length X1 of the one edge of the opening part having a rectangular shape, which is included in the second shaping member 22. Then, the control system 140 controls the exposing unit 150 so as to expose the region 208 which is determined by the length of the first edge Y and the length of the second edge X as shown in FIG. 2C.

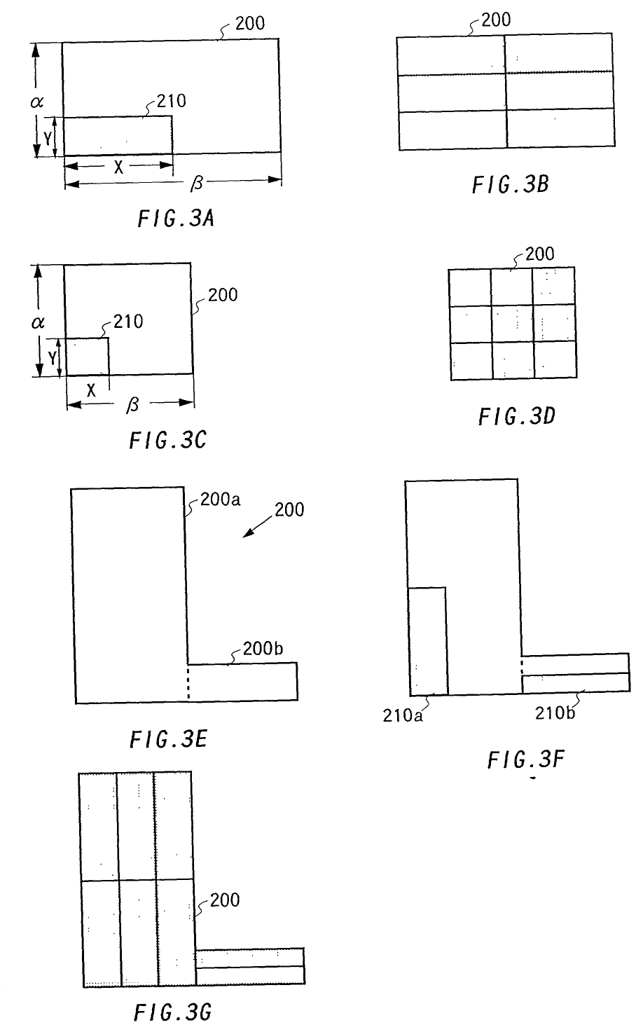

FIGS. 3A-3G show an example of an exposure pattern 200 and a cross sectional shape of the electron beam to be exposed on the wafer 44. As shown in FIG. 3A, the control system 140 may determine the length of the first edge Y included in the cross sectional shape 210 of the electron beam based on the length of the third edge a included in the exposure pattern 200 in the range where the deflection unit 60 deflects the electron beam and the fourth edge β, which is substantially perpendicular to the third edge.

Desirably, the control system 140 determines the cross sectional shape 210 of the electron beam to be exposed on the wafer 44 such that the number of shots of the electron beam necessary for exposing the exposure pattern 200 on the wafer 44 becomes the minimum. Specifically, the minimum number of shots for the exposure pattern 200 is calculated by dividing the area of the exposure pattern 200 by the area where the value of the electric current of the electron beam becomes the reference value or becomes less than the reference value, for example.

At this time, if the minimum number of shots includes the numerical value less than the decimal point, it is preferable to raise decimals less than the decimal point. Then, the control system 140 determines the length of the first edge Y included in the cross sectional shape 210 based on the length of the third edge a and the length of the fourth edge β included in the exposure pattern 200 so that the number of shots of the electron beam for the exposure pattern 200 becomes the minimum number of shots.

Furthermore, the length of the first edge of the cross sectional shape 210 may be determined based on the length of the edge in the longitudinal direction of the exposure pattern 200. Furthermore, the length of the second edge, which corresponds to the edge in the latitude direction of the exposure pattern 200, may be determined based on this length of the first edge. Then, the Control system 140 instructs the exposing unit 150 to expose the exposure pattern 200 on the wafer 44 based on the determined cross sectional shape 210 as shown in FIG. 3B. Furthermore, the cross sectional shapes 210 of the electron beam exposed a plurality of times in the exposure pattern 200 may all have a same shape and may have a different shape.

As shown in FIG. 3C, the control system 140 may determine the length of the first edge Y included in the cross sectional shape 210 so that the cross sectional shape 210 of the electron beam becomes similar to the exposure pattern 200 in the range where the electron beam is deflected by the deflection unit 60. At this time, it is preferable to determine the length of the first edge Y to be 1/N times, where N is natural number, of the length of the third edge α of the exposure pattern 200. Then, the control system 140 instructs the exposing unit 150 to expose the exposure pattern 200 on the wafer 44 based on the determined cross sectional shape 210 as shown in FIG. 3D.

As shown in FIG. 3E, the control system 140 may determine the cross sectional shape 210 of the electron beam by dividing the exposure pattern 200 in the range where the deflection unit 60 deflects the electron beam into the plurality of regions. The controller system 140 may then determine the cross sectional shape 210 of the electron beam for each of the regions as shown in FIG. 3F by the operation described in FIG. 3A. After the predetermined rectangular cross section 210 in the exposure pattern 200 is exposed, the unexposed region included in the exposure pattern 200 is recognized as the new exposure pattern. Then, the control system 140 may determine the cross sectional shape of the electron beam to be exposed on the wafer 44 for the new exposure pattern. Then, the control system 140 instructs the exposing unit 150 to expose the exposure pattern 210 on the wafer 44 based on the determined cross sectional shape 210 as shown in FIG. 3G.

The operation of the whole of the electron beam exposure apparatus 100 will be explained with referring to FIG. 1, FIG. 2, and FIG. 3. First, the electron guns 10 generate a plurality of electron beams. The electron beams generated by the electron guns 10 are irradiated to the first shaping member 14 so as to be shaped.

The first multi-axis electron lens 16 converges the plurality of electron beams, which are shaped in rectangular shape, independently. Also, the first multi-axis electron lens 16 adjusts the focus of each of the electron beams independently to the second shaping member 22. The first shaping deflection unit 18 and the second shaping deflection unit 20 deflect the electron beams so as to shape the cross sectional shape of the electron beams based on the instruction of the control system 140, which is a control means explained in FIG. 2 and FIG. 3.

The first shaping deflection unit 18 deflects each of the plurality of the electron beams, which are shaped in the rectangular shape, independently to the desired position of the second shaping member 22. The second shaping deflection unit 20 deflects each of the plurality of the electron beams, which are deflected by the first shaping deflection unit 18, independently in the substantially perpendicular direction to the second shaping member 22. The second shaping member 22, which includes a plurality of opening parts having a rectangular shape, further shapes the plurality of electron beams, which have a rectangular cross section that is irradiated to each of the opening parts, to the electron beams having a desired rectangular cross sectional shape that is to be irradiated to the wafer 44.

The second multi-axis electron lens 24 converges a plurality of electron beams independently and adjusts the focus of each of the electron beams to the blanking electrode array 26 independently. The electron beams, the foci of which are adjusted by the second multi-axis electron lens 24, pass a plurality of apertures included in the blanking electrode array 26.

The blanking electrode array control unit 86 controls the application of the voltage on the deflection electrode that is provided nearby to each of the apertures formed in the blanking electrode array 26. The blanking electrode array 26 switches whether or not to irradiate the electron beams on the wafer 44 based on the voltage applied on the deflection electrode. Furthermore, the blanking electrode array control unit 86 controls the time for irradiating the electron beams on the wafer 44 based on the irradiation time of the electron beams determined according to the cross sectional shape of the electron beams.

The electron beam, which is not deflected by the blanking electrode array 26, is reduced in its electron beam diameter by the third multi-axis electron lens 34 and passed through the opening part included in the electron beam shielding member 28. The fourth multi-axis electron lens 36 converges the plurality of electron beams independently and adjusts the focus of each of the electron beams to the deflection unit 60 independently. The electron beams, the foci of which are adjusted, are entered to the deflectors included in the deflection unit 60.

The deflection control unit 92 controls the plurality of deflectors included in the deflection unit 60 independently. The deflection unit 60 deflects each of the plurality of electron beams entered into the plurality of deflectors independently to the desired exposure position of the wafer 44. The foci of the plurality of electron beams that pass through the deflection unit 60 are adjusted to the wafer 44 by the fifth multi-axis electron lens 62, and the electron beams are irradiated to the wafer 44.

During the exposure process, the wafer stage control unit 96 moves the wafer-stage driving unit 48 in a constant direction. The blanking electrode array control unit 86 determines the apertures that pass through the electron beams based on the exposure pattern data and controls the power for each of the apertures. It becomes possible to expose the desired circuit pattern on the wafer 44 by changing the apertures, through which the electron beams pass, properly according to the movement of the wafer 44 and further by deflecting the electron beams by the deflection unit 60.

As clear from the above description, according to the present embodiment, the cross sectional shapes of the electron beams to be exposed on a wafer can be effectively determined for the pattern to be exposed on the wafer.

Although the present invention has been described by way of exemplary embodiments, it should be understood that many changes and substitutions may be made by those skilled in the art without departing from the spirit and the scope of the present invention which is defined only by the appended claims.

What is claimed is:

1. An electron beam exposure apparatus for exposing a wafer using an electron beam, comprising:
a shaping unit for shaping a cross sectional shape of said electron beam so that said cross sectional shape has a rectangular cross-section that includes a first edge and a second edge, which is substantially perpendicular to said first edge; and
a control unit connected to said shaping unit for determining at least one of a length of said second edge and an irradiation time of said electron beam based on a length of said first edge.

2. An electron beam exposure apparatus as claimed in claim 1, wherein said control unit determines at least one of said length of said second edge and said irradiation time of said electron beam further based on a pattern to be exposed on said wafer.

3. An electron beam exposure apparatus as claimed in claim 1, wherein said shaping unit has a shaping member that includes an opening part which shapes said cross-sectional shape of said electron beam to said rectangular cross-section; and
a maximum length of said second edge is limited by a size of said opening part.

4. An electron beam exposure apparatus as claimed in claim 1, wherein said control unit determines at least one of said length of said second edge and said irradiation time so that a product of a current density of said electron beam, which is shaped in said rectangular cross-section, an area of said rectangular cross-section, and said irradiation time becomes substantially constant.

5. An electron beam exposure apparatus as claimed in claim 1, wherein said control unit determines said length of said second edge so that a product of a current density of said electron beam, which is shaped in said rectangular cross-section, and an area of said rectangular cross-section becomes substantially constant.

6. An electron beam exposure apparatus as claimed in claim 2, further comprising a deflection unit that deflects said electron beam shaped in said rectangular cross-section; and
said pattern to be exposed on said wafer in a range where said deflection unit deflects said electron beam, which is shaped in said rectangular cross-section, includes a third edge and a fourth edge, which is substantially perpendicular to said third edge; and
said length of said first edge is determined based on a length of said third edge and a length of said fourth edge.

7. An electron beam exposure apparatus as claimed in claim 6, wherein a length of an edge along a longitudinal direction of said rectangular cross-section is limited by the size of said opening part.

8. An electron beam exposure apparatus as claimed in claim 6, wherein said control unit has a means for determining said length of said first edge so that the shape of said rectangular cross-section becomes similar to the shape of said pattern to be exposed on said wafer.

9. An electron beam exposure apparatus as claimed in claim 6, wherein said control unit has a means for determining said length of said first edge to divide said pattern to be exposed on said wafer in a range where said deflection unit deflects said electron beam so that the number of times of irradiating said shaped electron beam becomes minimum.

10. An electron beam exposure apparatus as claimed in claim 1, further comprising a means for generating a plurality of said electron beams; and
said shaping unit shapes a cross-sectional shape of each of said electron beams into the rectangular cross-section that includes said first edge and said second edge, which is substantially perpendicular to said first edge; and
said control unit determines at least one of each of said lengths of said second edges and each of said irradiation times of each said electron beams.

11. A method for exposing a wafer using an electron beam, comprising:
shaping a cross sectional shape of said electron beam so that said cross sectional shape has a rectangular cross-section that includes a first edge and a second edge, which is substantially perpendicular to said first edge; and
determining at least one of a length of said second edge and an irradiation time of said electron beam based on a length of said first edge.

12. A method as claimed in claim 11, wherein said determining determines at least one of said length of said second edge and said irradiation time of said electron beam further based on a pattern to be exposed on said wafer.

13. A method as claimed in claim 11, wherein said shaping shapes said cross-sectional shape of said electron beam to said rectangular cross-section by an opening part having an opening, through which said electron beam is passed through; and
a maximum length of said second edge is limited by a size of said opening part.

14. A method as claimed in claim 11, wherein said determining determines at least one of said length of said second edge and said irradiation time so that a product of a current density of said electron beam, which is shaped in said rectangular cross-section, an area of said rectangular cross-section, and said irradiation time becomes substantially constant.

15. A method as claimed in claim 11, wherein said determining determines said length of said second edge so that a product of a current density of said electron beam, which is shaped in said rectangular cross-section, and an area of said rectangular cross-section becomes substantially constant.

16. A method as claimed in claim 12, further comprising:
deflecting said electron beam shaped in said rectangular cross-section; and
said pattern to be exposed on said wafer in a range, where said electron beam shaped in said rectangular cross-section is deflected, includes a third edge and a fourth edge, which is substantially perpendicular to said third edge; and
said length of said first edge is determined based on a length of said third edge and a length of said fourth edge.

17. A method as claimed in claim 16, wherein a length of an edge along a longitudinal direction of said rectangular cross-section is limited by the size of said opening part.

18. A method as claimed in claim 16, wherein said determining determines said length of said first edge so that the shape of said rectangular cross-section becomes similar to the shape of said pattern to be exposed on said wafer.

19. A method as claimed in claim 16, wherein said determining determines said length of said first edge to divide said pattern to be exposed on said wafer in a range where said electron beam is deflected so that the number of times of irradiating said shaped electron beam becomes minimum.

20. A method as claimed in claim 11, further comprising:
generating a plurality of said electron beams; and
said shaping shapes a cross-sectional shape of each of said electron beams into the rectangular cross-section that includes said first edge and said second edge, which is substantially perpendicular to said first edge; and
said determining determines at least one of each of said lengths of said second edges and each of said irradiation times of each said electron beams.
